SEQUENCE LISTING (43 sequences)

SEQ ID NO: 1 - DNA, 31 bp, homo sapiens
tccctggtggtctagtggttaggattcggca

SEQ ID NO: 2 - RNA, 23 nt, homo sapiens
caaagugcuguucgugcagguag

SEQ ID NO: 3 - DNA, 21 bp, homo sapiens
aucacauugccagggauuucc

SEQ ID NO: 4 - DNA, 21 bp, homo sapiens
uucacaguggcuaaguuccgc

SEQ ID NO: 5 - DNA, 21 bp, homo sapiens
cauaaaguagaaagcacuacu

SEQ ID NO: 6 - DNA, 24 bp, homo sapiens
ccccccactgctaaatttgactgg

SEQ ID NO: 7 - DNA, 25 bp, homo sapiens
cccccactgctaaatttgactggtt

SEQ ID NO: 8 - DNA, 31 bp, homo sapiens
agcccggctagctcagtcggtagagcatgag

SEQ ID NO: 9 - DNA, 23 bp, homo sapiens
ccccccactgctaaatttgactg

SEQ ID NO: 10 - DNA, 30 bp, homo sapiens
gcccggctagctcagtcggtagagcatgag

SEQ ID NO: 11 - DNA, 25 bp, homo sapiens
ccccccactgctaaatttgactggc

SEQ ID NO: 12 - DNA, 25 bp, homo sapiens
ccccccactgctaaatttgactggt

SEQ ID NO: 13 - DNA, 25 bp, homo sapiens
ccccccactgctaaatttgactggt

SEQ ID NO: 14 - DNA, 31 bp, homo sapiens
taaagtgctgacagtgcagatagtggtcctc

SEQ ID NO: 15 - DNA, 31 bp, homo sapiens
gtttccgtagtgtagtggtcatcacgttcgc

SEQ ID NO: 16 - DNA, 31 bp, homo sapiens
ttccctggtggtctagtggttaggattcggc

SEQ ID NO: 17 - DNA, 27 bp, homo sapiens
gaggaatgatgacaagaaaaggccgaa

SEQ ID NO: 18 - DNA, 29 bp, homo sapiens
tctgctgcctcagcctcccgagtagctga

SEQ ID NO: 19 - DNA, 29 bp, homo sapiens
tactcaggaggctgaggcaggagaatggc

SEQ ID NO: 20 - DNA, 31 bp, homo sapiens
tcctgggttcaggtgattctcctgcctcagt

SEQ ID NO: 21 - DNA, 29 bp, homo sapiens
tagtcccagctacttgggaggctgaggca

SEQ ID NO: 22 - DNA, 32 bp, homo sapiens
gaccagcctggccaacatggtgaaaccccgcc

SEQ ID NO: 23 - DNA, 31 bp, homo sapiens
tgcctcccggattcaagtgattctcctgcct

SEQ ID NO: 24 - DNA, 30 bp, homo sapiens
tcccagctacctaggaggctgaggcaggag

SEQ ID NO: 25 - DNA, 29 bp, homo  sapiens
tactcaggaggctgagacaggagaattgc

SEQ ID NO: 26 - DNA, 32 bp, homo sapiens
atcgaggctagagtcacgcttgggtatcggct

SEQ ID NO: 27 - DNA, 31 bp, homo sapiens
gcctgggtagctcagtcggtagagcatcaga

SEQ ID NO: 28 - DNA, 31 bp, homo sapiens
ggtcgctggttcgaatccggctcgaaggacc

SEQ ID NO: 29 - DNA, 30 bp, homo sapiens
gcccggatgatcctcagtggtctggggtgc

SEQ ID NO: 30 - DNA, 30 bp, homo sapiens
taaggtgcatctagtgcagatagtgaagta

SEQ ID NO: 31 - DNA, 28 bp, homo sapiens
ggaggtgatgaactgtctgagcctgacc

SEQ ID NO: 32 - DNA, 30 bp, homo sapiens
cccctggtggtctagtggttaggattcggc

SEQ ID NO: 33 - DNA, 30 bp, homo sapiens
gttcactgatgagagcattgttctgagcca

SEQ ID NO: 34 - DNA, 27 bp, homo sapiens
ttcactgatgagagcattgttctgagc

SEQ ID NO: 35 - DNA, 31 bp, homo sapiens
tccgatcctcgttgttttggctatggccaga

SEQ ID NO: 36 - DNA, 30 bp, homo sapiens
ctgacctcaagtgatccacctgcctcagcc

SEQ ID NO: 37 - DNA, 26 bp, homo sapiens
cccccactgctaaatttgactggcta

SEQ ID NO: 38 - DNA, 25 bp, homo sapiens
cccccactgctaaatttgactggct

SEQ ID NO: 39 - DNA, 30 bp, homo sapiens
ggccgtgatcgtatagtggttagtactctg

SEQ ID NO: 40 - DNA, 26 bp, homo sapiens
ccccccactgctaaatttgactggtt

SEQ ID NO: 41 - DNA, 32 bp, homo sapiens
tgaggtagtaggttgtatggtttagagttaca

SEQ ID NO: 42 - DNA, 27 bp, homo sapiens
ccccccactgctaaatttgactggcta

SEQ ID NO: 43 - DNA, 26 bp, homo sapiens
ccccccactgctaaatttgactggct

(19) United States
(12) Patent Application Publication
(10) Pub. No.: US 2021/0130904 A1
(43) Pub. Date: May 6, 2021

(54) METHOD OF DIAGNOSIS OF COLORECTAL CANCER

(71) Applicants: MASARYKOVA UNIVERZITA, Brno (CZ); MASARYKUV ONKOLOGICKY USTAV, Brno (CZ)

(72) Inventors: Ondrej SLABY, Brno (CZ); Petra VYCHYTILOVA, Brno (CZ); Marek SVOBODA, Syrovice (CZ); Milana SACHLOVA, Brno (CZ)

(73) Assignees: MASARYKOVA UNIVERZITA, Brno (CZ); MASARYKUV ONKOLOGICKY USTAV, Brno (CZ)

(21) Appl. No.: 16/629,520

(22) PCT Filed: Jul. 13, 2018
(86) PCT No.: PCT/CZ2018/050037

§ 371 Date: Jan. 8, 2020

(30) Foreign Application Priority Data
Jul. 17, 2017 (EP) 17181646.5

(51) Int. Cl. C12Q 1/6886

(52) U.S. Cl. CPC C12Q 1/6886; C12Q 2600/178; C12Q 2600/158; C12Q 2600/118

(57) ABSTRACT

Method of diagnosing and prognosing colorectal cancer using piRNA-hsa-5937, having the sequence TCCCTGGTGGTCTAGTGGTTAGGATTCGGCA (SEQ ID NO. 1), as a biomarker, in combination with at least one miRNA selected from:

(SEQ ID NO. 3)
miR-23a-3p, having the sequence
AUCACAUUGCCAGGGAUUUCC,
(SEQ ID NO. 4)
miR-27a-3p, having the sequence
UUCACAGUGGCUAAGUUCCGC,
(SEQ ID NO. 5)
miR-142-5p, having the sequence
CAUAAAGUAGAAAGCACUACU.

The resulting method uses a body fluid as the input, is non-invasive, has a high sensitivity and specificity even in very early stages of the disease, and is cost-effective.

### FIELD OF ART

The present invention relates to a method of diagnosis of colorectal cancer from body fluid samples, using piRNAs and miRNAs as biomarkers.

### BACKGROUND ART

Colorectal cancer (CRC) accounts for 9.7% of all cancers, it is the third most common cancer worldwide and the fourth most common cause of cancer related deaths, accounting for more than 13% of all cancer deaths and representing the second leading cause of malignant mortality in industrialized countries. The survival rate is significantly better for early diagnosed patients. The chance of surviving CRC is closely related to the stage of the disease at diagnosis; the earlier the diagnosis, the greater the likelihood of survival. For example, there is less than a 20% chance of 5-year survival when diagnosed late in the disease timeframe (stage IV), while there is greater than 90% chance of 5-year survival when diagnosed early (stage I). The early diagnosis usually leads to possibility of curative surgical intervention which is feasible only for patients with disease limited to primary tumor and regional lymph nodes.

Most colorectal cancers develop slowly, beginning as small benign colorectal adenomas which progress over several decades to larger and more dysplastic lesions which eventually become malignant. This gradual progression provides multiple opportunities for prevention and intervention. As early diagnosis offers the best chance for a curative treatment, CRC patients would greatly benefit from early detection because of the effectiveness of surgical treatment early on.

Unfortunately, most cases of CRC are still diagnosed in the already advanced stages when a curative surgical treatment is not possible, and chemotherapy remains the only option in spite of high costs and undesirable side effects. Because of the absence of efficient diagnostic methods of CRC, patients with colorectal neoplasia would most benefit from efficient early diagnostic CRC tests that can reveal early stages of CRC, thereby enabling preventive interventions.

The risk of CRC begins to increase after the age of 50; thereafter the risk continues to rise, approximately doubling with each succeeding decade. Increased risk is slower in women and, before age 75, women have a lower incidence of CRC than men.

The most common and earliest detection procedures available at present for CRC are (i) the fecal occult blood test (FOBT), which is based on the assumption that cancers will bleed, and can therefore be detected in the stool using chemical or immunological assays; significant tumor size must typically exist before fecal blood is detected; (ii) imaging methods such as virtual colonoscopy; and (iii) invasive methods that identify gross abnormalities such as sigmoidoscopy or colonoscopy.

The FOBT is the most widespread clinically useful test used for CRC, and involves a crude test for the peroxidase-like activity of heme in hemoglobin (guayac test) or a specific antibody against hemoglobin (Faecal Immunochemical testing, FIT). However, this screening technique suffers from a certain number of handicaps: The major drawback is its mediocre sensitivity of approximately 50%, with 20% sensitivity for adenomas (which, if they are large in size, will result in the development of cancer in 1 case out of 10), due to the fact that not all adenomas and CRCs bleed. The test is also not very specific as the appearance of blood in the stools may be related to a non-tumour condition (e.g. ulcerative colitis, hemorrhoids, fistulae, etc.). Thus, a colonoscopy must also be carried out, with the drawbacks described below.

Computerized Tomography Colonography (CTC), or virtual colonoscopy, is a recent non-invasive technique for imaging the colon, with reports varying dramatically on the performance characteristics of the assay (ranging between 39% and 94% specificity), due primarily to technological differences in the patient preparation and the hardware and software used for the analysis. Other limitations of CTC include high false-positive readings, inability to detect flat adenomas, no capacity to remove polyps, repetitive and cumulative radiation doses, and cost.

Invasive methods for identifying gross abnormalities include sigmoidoscopy and colonoscopy. Colonoscopy is usually the preferred method for screening average and increased-risk individuals over the age of 50 who have a history of CRC or prior adenomatous polyps, or other predisposing diseases such as inflammatory bowel disease. Although colonoscopy is still the standard test for the presence or absence of polyps and CRC, it can miss up to 15% of lesions>1 cm in diameter. Complications with colonoscopy can include perforation, hemorrhage, respiratory depression, arrhythmias, and infection. Approximately one in 1,000 patients suffers perforations and three in 1,000 experience hemorrhaging. Between one and three deaths out of 10,000 tests occur as a result of the procedure. Other disadvantages such as the lack of trained personnel, patient discomfort, and high cost and the low level of compliancy (around 2%) for colonoscopy will likely prevent the colonoscopy from becoming a routine CRC screening method for the general population. Most sporadic CRCs are thought to develop from benign adenomas, of which only a small number will ever develop to malignancy. Given that the time period for malignant development from benign adenoma is five to ten years, the detection of adenomas across the general population by colonoscopy would require a gross overtreatment of patients, being both costly and potentially harmful. Colonoscopy is the next test after a positive FOBT, and with a 60% false positive rate, imposes unnecessary risks that require improvement.

Therefore, biomarkers enabling early diagnosis, preferably also prognosis, and later on accurate monitoring of patients' progression and response to treatment are highly needed. Biomarkers present in samples that can be obtained from the patients by low-invasive or non-invasive methods (such as blood samples) are preferred, as they can be used in population screening.

Currently, there is no serological screening test or specific diagnostic test which detects early CRC. Warren et al (BMC Med 9 (2011) 133) have recently described Septin 9 methylation as a sensitive and specific blood test for colorectal cancer. Although there is a commercial test it is not routinely used for screening, since this test is labour intensive and expensive.

In the recent years a large amount of so-called CRC specific genes has been reported. The vast majority of the corresponding research papers or patent applications are based on data obtained by analysis of RNA expression patterns in colon cancer tissue versus a different tissue, an adjacent normal tissue or healthy colon tissue. Such approaches may be summarized as differential mRNA display techniques. However, differences on the level of mRNA are not necessarily mirrored by the level of the corresponding proteins. A protein encoded by a rare mRNA may be found in very high amounts and a protein encoded by an abundant mRNA may nonetheless be hard to detect and find at all. This lack of correlation between mRNA-level and protein level is due to reasons like mRNA stability, efficiency of translation, stability of the protein, etc.

There also are recent approaches investigating the differences in protein patterns between different tissues or between healthy and diseased tissue in order to identify candidate marker molecules which might be used in the diagnosis of CRC. Bruenagel et al. (Cancer Research 62 (2002) 2437-2442), have identified seven nuclear matrix proteins which appear to be more abundant in CRC tissue as compared to adjacent normal tissue. Further, WO 2010/061996 A1 discloses several protein biomarkers, some which can be detected in serum of CRC patients.

Other representative examples known as molecular markers of colorectal cancer are as follows: WO 2005/015224 discloses a method to diagnose colorectal cancer using an antibody against protein RLA-0 (60S acidic ribosomal protein P0). WO 2004/079368 discloses that HSP90 is highly expressed in colorectal cancers. WO 2004/071267 describes a method for diagnosing colorectal cancer in an early stage by measuring NNMT (nicotinamide N-methyltransferase) in a stool sample, and WO 2005/015234 discloses that SAHH (S-adenosylhomocysteine hydrolase) protein is able to be utilized in the diagnosis of colorectal cancer. In addition, U.S. Pat. No. 7,501,243 discloses TTK (Tyrosine threonine kinase) as a colon cancer marker.

However, despite candidate protein markers being proposed, to date their clinical/diagnostic utility has not been demonstrated. Ideally, a new diagnostic marker as a single marker should also lead to a progress in diagnostic sensitivity and/or specificity either if used alone or in combination with one or more other markers, compared to currently used diagnostic methods. At present, for example, diagnostic blood tests based on the detection of carcinoembryonic antigen (CEA) or CA19-9 are available to assist diagnosis in the field of CRC. The development of the first assays of serum carcinoembryonic antigen (CEA) in 1965 raised considerable hope on the possibility of screening test in blood. However, subsequent work showed that the sensitivity of CEA was less than 35% in individuals with invasive cancer; it could not detect early forms and its specificity was insufficient, since its serum levels increase in several pathologies. The limitations of the CA19-9 antigen, introduced more recently, are comparable to those of CEA. The current use of these two molecules is limited to post-therapeutic surveillance. Moreover, these biomarker is expressed by other types of cancer, and in benign pathologies. Despite everything, it is possible to increase sensitivity without losing specificity by combining, with CEA, another tumor marker.

Recently, signature panels of circulating miRNAs allowing a non-invasive early detection and prognosis prediction of colon cancer from a body fluid (in particular, blood serum) of the patients were reported in Vychytilova-Faltejskova et al., Carcinogenesis, 2016, 941-950. The diagnostic panel comprised miR-23a-3p, miR-27a-3p, miR-142-5p, miR-276c-3p and showed sensitivity of 89% and specificity of 81% in the independent validation. The prognostic panel comprised miR-23a-3p, miR-276c-3p. However, some members of this diagnostic and prognostic panel were described as being involved with other solid cancers than colorectal cancer, and thus their specificity in population screening might be compromised.

Taken together, above mentioned diagnostic approaches suffer from low sensitivity in detecting early stages (I and II) of colorectal cancer and no definite biomarkers have been identified to date that can be reliably used for detecting CRC in blood samples. Thus, there is a clinical need for identifying specific biomarkers for early detection of CRC that can be tested in a noninvasive manner

The present invention aims at identifying body fluid biomarkers suitable for diagnosing and prognosing CRC which could be used in screening tests and would be relevant also in early stages of the disease, to increase the survival rates. The desirable tests would comprise as many as possible of the following features: average-risk, asymptomatic individuals, highly sensitive, non-invasive, low-risk, cost-effective, and ease of implementation across a large population.

### DISCLOSURE OF THE INVENTION

The present invention provides newly identified piRNAs which are useful as colorectal cancer biomarkers. piRNAs (or PIWI-interacting RNAs) are small non-coding RNA molecules expressed in eukaryotic cells. They are distinct from miRNAs in size (26-31 nucleotides rather than 21-24 nucleotides), lack of sequence conservation and increased complexity. They seem to be more specific markers than miRNAs.

The newly identified piRNAs are piRNA-hsa-5937 (or piR-5937, alias piR-43771), having the sequence TCCCTGGTGGTCTAGTGGTTAGGATTCGGCA (SEQ ID NO. 1), and piR-28876, having the sequence GTTTCCGTAGTGTAGTGGTCATCACGTTCGC (SEQ ID NO. 15). Both piRNAs are useful as colorectal cancer biomarkers which are downregulated in cancer patients.

Furthermore, said piRNAs are used in biomarker combination with other piRNAs or miRNAs. The expression levels of circulating piRNAs and miRNAs are used, and the diagnostic and/or prognostic methods are carried out using samples of body fluids from patients. The body fluids that can be used include in particular blood, blood serum, blood plasma, urine and saliva.

Therefore, in a first aspect of the present invention, a method for diagnosing colorectal cancer is provided, wherein the level of expression of piRNA-hsa-5937 is determined in a sample of body fluid, then the level of expression of piRNA-hsa-5937 in the sample is compared with a level of expression of piRNA-hsa-5937 in the body fluid of a healthy human, whereas when the level of expression of piRNA-hsa-5937 in the sample is lower than the level of expression of piRNA-hsa-5937 in the body fluid of a healthy human, colorectal cancer is diagnosed in the sample.

The method for diagnosing colorectal cancer using piRNA-hsa-5937 as a marker further includes determining the levels of expression in the body fluid sample of at least one miRNA selected from:

(SEQ ID NO. 3)
miR-23a-3p, having the sequence
AUCACAUUGCCAGGGAUUUCC,
(SEQ ID NO. 4)
miR-27a-3p, having the sequence
UUCACAGUGGCUAAGUUCCGC,
(SEQ ID NO. 5)
miR-142-5p, having the sequence
CAUAAAGUAGAAAGCACUACU,

comparing them with the levels of expression of the same miRNAs in body fluid of a healthy human, whereas when the the level of expression of said miRNAs in the sample is higher than the level of expression of said miRNAs in body fluid of a healthy human, colorectal cancer is diagnosed in the sample.

The level of expression in body fluid of a healthy human is obtained by establishing the level of expression in body fluid samples obtained from healthy volunteers not suffering from colorectal cancer. The average level of expression can be obtained by known statistical methods if desired.

In order to compare the levels of expression, it is advantageous when the content of piRNA-hsa-5937 in the sample is referenced to a substance having a constant content in body fluid (i.e., normalized). Such substance may be miR-93-5p, having the sequence CAAAGUGCUGUUCGUGCAGGUAG (SEQ ID NO. 2).

It is also advantageous when the content of the above-defined miRNAs in the sample is referenced to a substance having a constant content in body fluid (i.e., a normalizing marker). Preferably, the normalizing marker is miR-93-5p.

In one embodiment, diagnostic score (DxScore) can be calculated from the levels of expression, or preferably from the normalized levels of expression of the biomarkers. Value of the diagnostic score can then be used for classifying the patients into patients suffering from colorectal cancer or patients not suffering from colorectal cancer.

If the DxScore is higher than a cut-off value, the sample is positive (colorectal cancer is diagnosed), and if the DxScore is lower than a cut-off value, the sample is negative (colorectal cancer is not diagnosed). The cut-off value can be obtained using known methodologies, e.g. a statistical analysis of the normalized body fluid levels in groups of healthy volunteers (controls) and colorectal cancer patients.

Using the DxScore, the method allows to achieve the sensitivity of 96.25% and specificity of 97.50%.

In one embodiment, normalized serum level of hsa-miR-23a in the sample is calculated as X(miR-23a)=2^{-(Ct.miR23a-Ct.miR93)}, normalized serum level of hsa-miR-27a in the sample is calculated as X(miR-27a)=2^{-(Ct.miR27a-Ct.miR93)}, normalized serum level of hsa-miR-142-5p in the sample is calculated as X(miR-142-5p)=2^{-(Ct.miR142-5p-Ct.miR93)}, normalized serum level of piRNA-hsa-5937 in the sample is calculated as X(piR-5937)=2^{-(Ct.piR5937-Ct.miR93)}.

The following formula can then be used for calculating the diagnostic score (DxScore):

DxScore=−0.8895+62.92648*X(miR-23a)−8.08928*X(miR-27a)+73.16745*X(miR-142-5p)−1.6757*X(piR-5937).

The normalized levels of the biomarkers and diagnostic score (DxScore) may be used, Preferably, the normalizing marker is miR-93-5p. If the normalized levels or the DxScore are lower than a pre-determined cut-off value then the sample is positive (colorectal cancer is diagnosed). If the normalized level levels or the DxScore are higher than a pre-determined cut-off value then the sample is negative (colorectal cancer is not diagnosed). The pre-determined cut-off value and/or the DxScore can be obtained and determined using known methodologies, e.g. a statistical analysis of the normalized body fluid levels in groups of healthy volunteers (controls) and colorectal cancer patients.

Yet further, the method for diagnosing colorectal cancer may further include determining of levels of expression in the body fluid sample of at least one of the following piRNAs, comparing them with the levels of expression of the same piRNA, respectively, in body fluid of a healthy human, whereas when the level of expression of said piRNA shows the change determining colorectal cancer relative to the level of expression of the same piRNA in body fluid of a healthy human, colorectal cancer is diagnosed in the sample:

| | | Change in |
|---|---|---|
| | | expression |
| | | level |
| | | determining |
| | | colorectal |
| piRNA | Sequence | cancer |
| piR-hsa-32161 | CCCCCCACTGCTAAATTTGACTGG (SEQ ID NO. 6) | decrease |
| piR-hsa-32238 | CCCCCACTGCTAAATTTGACTGGTT (SEQ ID NO. 7) | decrease |
| piR-hsa-1242 | AGCCCGGCTAGCTCAGTCGGTAGAGCATGAG (SEQ ID NO. 8) | decrease |
| piR-hsa-32187 | CCCCCCACTGCTAAATTTGACTG (SEQ ID NO. 9) | decrease |
| piR-hsa-27620 | GCCCGGCTAGCTCAGTCGGTAGAGCATGAG (SEQ ID NO. 10) | decrease |
| piR-hsa-32162 | CCCCCCACTGCTAAATTTGACTGGC (SEQ ID NO. 11) | decrease |
| piR-hsa-32195 | CCCCCCACTGCTAAATTTGACTGGT (SEQ ID NO. 12) | decrease |
| piR-hsa-32158 | CCCCCCACTGCTAAATTTGACTGGT (SEQ ID NO. 13) | decrease |
| piR-hsa-29218 | TAAAGTGCTGACAGTGCAGATAGTGGTCCTC(SEQ ID NO. 14) | decrease |
| piR-hsa-28876 | GTTTCCGTAGTGTAGTGGTCATCACGTTCGC (SEQ ID NO. 15) | decrease |
| piR-hsa-24672 | TTCCCTGGTGGTCTAGTGGTTAGGATTCGGC (SEQ ID NO. 16) | decrease |
| piR-hsa-26872 | GAGGAATGATGACAAGAAAAGGCCGAA (SEQ ID NO. 17) | increase |
| piR-hsa-8226 | TCTGCTGCCTCAGCCTCCCGAGTAGCTGA (SEQ ID NO. 18) | increase |
| piR-hsa-30715 | TACTCAGGAGGCTGAGGCAGGAGAATGGC (SEQ ID NO. 19) | increase |
| piR-hsa-6746 | TCCTGGGTTCAGGTGATTCTCCTGCCTCAGT (SEQ ID NO. 20) | increase |
| piR-hsa-30937 | TAGTCCCAGCTACTTGGGAGGCTGAGGCA (SEQ ID NO. 21) | increase |
| piR-hsa-26543 | GACCAGCCTGGCCAACATGGTGAAACCCCGCC (SEQ ID NO.22) | increase |
| piR-hsa-15278 | TGCCTCCCGGATTCAAGTGATTCTCCTGCCT (SEQ ID NO. 23) | increase |
| piR-hsa-5505 | TCCCAGCTACCTAGGAGGCTGAGGCAGGAG (SEQ ID NO. 24) | increase |
| piR-hsa-30714 | TACTCAGGAGGCTGAGACAGGAGAATTGC (SEQ ID NO. 25) | increase |
| piR-hsa-1359 | ATCGAGGCTAGAGTCACGCTTGGGTATCGGCT (SEQ ID NO.26) | decrease |
| piR-hsa-27139 | GCCTGGGTAGCTCAGTCGGTAGAGCATCAGA (SEQ ID NO. 27) | decrease |
| piR-hsa-28488 | GGTCGCTGGTTCGAATCCGGCTCGAAGGACC (SEQ ID NO. 28) | decrease |
| piR-hsa-27616 | GCCCGGATGATCCTCAGTGGTCTGGGGTGC (SEQ ID NO. 29) | decrease |
| piR-hsa-29716 | TAAGGTGCATCTAGTGCAGATAGTGAAGTA (SEQ ID NO. 30) | decrease |
| piR-hsa-28019 | GGAGGTGATGAACTGTCTGAGCCTGACC (SEQ ID NO. 31) | decrease |
| piR-hsa-23231 | CCCCTGGTGGTCTAGTGGTTAGGATTCGGC (SEQ ID NO. 32) | decrease |
| piR-hsa-28846 | GTTCACTGATGAGAGCATTGTTCTGAGCCA (SEQ ID NO. 33) | decrease |
| piR-hsa-24360 | TTCACTGATGAGAGCATTGTTCTGAGC (SEQ ID NO. 34) | decrease |
| piR-hsa-6046 | TCCGATCCTCGTTGTTTTGGCTATGGCCAGA (SEQ ID NO. 35) | decrease |
| piR-hsa-23940 | CTGACCTCAAGTGATCCACCTGCCTCAGCC (SEQ ID NO. 36) | increase |
| piR-hsa-32182 | CCCCCACTGCTAAATTTGACTGGCTA (SEQ ID NO. 37) | decrease |
| piR-hsa-32167 | CCCCCACTGCTAAATTTGACTGGCT (SEQ ID NO. 38) | decrease |
| piR-hsa-28190 | GGCCGTGATCGTATAGTGGTTAGTACTCTG (SEQ ID NO. 39) | decrease |
| piR-hsa-23210 | CCCCCCACTGCTAAATTTGACTGGTT (SEQ ID NO. 40) | decrease |
| piR-hsa-11362 | TGAGGTAGTAGGTTGTATGGTTTAGAGTTACA (SEQ ID NO.41) | decrease |
| piR-hsa-23209 | CCCCCCACTGCTAAATTTGACTGGCTA (SEQ ID NO. 42) | decrease |
| piR-hsa-32159 | CCCCCCACTGCTAAATTTGACTGGCT (SEQ ID NO. 43) | decrease |

All miRNAs and piRNAs used in the present invention can be referenced to the same substance having a constant content in body fluid, such as miR-93-5p.

In one embodiment, diagnostic score (DxScore) can be calculated from the levels of expression, or preferably from the normalized levels of expression of the biomarkers. Value of the diagnostic score can then be used for classifying the patients into patients suffering from colorectal cancer or patients not suffering from colorectal cancer.

The pre-determined cut-off value and/or the DxScore can be obtained and determined using known methodologies, e.g. a statistical analysis of the normalized body fluid levels in groups of healthy volunteers (controls) and colorectal cancer patients.

The present diagnostic method allows to diagnose colorectal cancer even in a very early stage (I and II clinical stage), which strongly increases the chances of successful treatment for the patients. It may be carried out with only one piRNA, and its sensitivity and specificity may be further improved by addition of further markers. The sensitivities and specificities achieved for early stage samples indicate that the present diagnostic method is very suitable for use in screening of population. In contrast to diagnostic methods used currently in the clinical practice, the method of the present invention is non-invasive, increases the patient comfort, and does not require complex equipment for taking the sample from the patient. These benefits will result in more people willing to participate in the screening, and in earlier detection of the colorectal cancer which greatly improves the chances of recovery and complete healing of the patients.

The CRC-specific biomarkers of the invention are circulating piRNAs and miRNAs, i.e., their levels in body fluids are indicative of diagnosis and prognosis of CRC. A test based on these biomarkers can be widely accepted by the general population as it is minimally invasive and can be used to monitor an individual's susceptibility to disease prior to resorting to, or in combination with, conventional screening methods. This will be extremely beneficial in the management of CRC risk, prevention, and treatment.

From this perspective, detection of small non-coding RNAs (microRNAs and PIWI-interacting RNAs) presents a very promising diagnostic approach.

MicroRNAs (miRNAs) are important regulators of gene expression comprising an abundant class of endogenous, small noncoding RNAs (18-25 nucleotides in length). They are capable of either promoting mRNA degradation or attenuating protein translation. Bioinformatic studies have estimated that miRNAs may regulate more than 50% of all human genes and each miRNA can control hundreds of gene targets. Some miRNAs are expressed in a cell-specific, tissue-specific and/or developmental stage-specific manner, while others are expressed ubiquitously. The number of verified miRNAs is still growing—the latest version of web-based database miRBase has annotated over 1800 precursor and 2342 mature sequences in the human genome. Based on the annotations for the genomic position of miRNAs which indicated that a vast majority of miRNAs is located in intergenic regions (>1 kb away from annotated or predicted genes), it has been postulated that most miRNA genes are transcribed as autonomous transcription units.

MiRNAs may serve as master regulators of many fundamental biological processes, such as embryogenesis, organ development, cellular differentiation, proliferation, apoptosis, etc., affecting such major biological systems as sternness and immunity.

When compared to mRNAs, microRNAs represent far more suitable biomarkers due to their long half-life, high stability under conditions that include RNase exposure, extremes of pH, long-term storage, and multiple freeze-thaw cycles.

Piwi-interacting RNA (piRNA) is the largest class of small non-coding RNA molecules expressed in animal cells. piRNAs form RNA-protein complexes through interactions with piwi proteins. They are distinct from microRNA (miRNA) in size (26-31 nt rather than 21-24 nt), lack of sequence conservation, and increased complexity. These small RNAs regulate gene expression on transcriptional and post-transcriptional level, however, they are preferentially involved in silencing of transponable elements LINE and SINE and thus contributing to genomic stability. Further, piRNAs participate also in other important biological processes, such as gametogenesis, chromosome segregation, or stem cell self-renewal. Although their expression has been firstly described in germ line cells, it has been proved that they are present in all tissue types and their expression is highly tissue-specific. In addition, the deregulated expression of piRNAs in tumor tissues has been described. Nevertheless, the exact function of these molecules in cancerogenesis is not known. Recently, free circulating piRNAs have been reported to be stably present in body fluids.

In a second aspect of the invention, a method of prognosis of overall survival of the patient is provided, wherein the level of expression of piRNA-hsa-5937 is determined in a sample of body fluid, then the level of expression of piRNA-hsa-5937 is compared with a reference expression level, whereas the level of expression of piRNA-hsa-5937 in the sample which is higher than the reference expression level indicates a favourable overall survival prognosis, while the level of expression of piRNA-hsa-5937 in the sample which is lower than the reference expression level indicates a poor overall survival prognosis.

The reference expression level can be obtained from a statistical analysis of samples from a group of colorectal cancer patients with favourable overall survival (for example at least more than 3 years from diagnosis) time and a group of colorectal cancer patients with poor overall survival time (for example less than 3 years from diagnosis). The methods of obtaining and assessing the data, and of performing the statistical analysis are known to the skilled person. For example, Receiver Operating Characteristics (ROC) analysis method may be used for identifying the reference expression level.

The favourable overall survival prognosis means a prognosis of overall survival of at least a predetermined number of years or months, for example a prognosis of overall survival of at least 20 or 24 or 26 months or 3 years from diagnosis.

The poor overall survival prognosis means a prognosis of overall survival of less than a predetermined number of years or months, for example a prognosis of overall survival of less than 20 or 24 or 26 months or 3 years from diagnosis.

In order to compare the levels of expression, it is advantageous when the content of piRNA-hsa-5937 in the sample is referenced to a substance having a constant content in body fluid, such as miR-93-5p.

Detection of Biomarkers:

The detection of piRNA and miRNA biomarkers of the present invention may be carried out by known methods, such as a real-time PCR assay, a micro-array assay, histochemistry assay, an immunological assay or sequencing assay.

Both miRNAs and piRNAs can be detected by standard real-time RT (Reverse-transcription) PCR technologies available at hospital-level laboratory medicine departments. The main advantages of real-time RT-PCR in comparison to serological tests is its high sensitivity, reliability and specificity. These days the costs of individual PCR miRNA/piRNA assays is low enough to enable its population-wide application in the screening programs Commercial assays are available to detect miRNAs/piRNAs by real-time RT-PCR (e.g. Thermo Fischer Scientific).

In RT-PCT methods, reverse transcription (RT) is first utilized to convert the target RNA into its cDNA, which is then subsequently amplified and quantified via one of several conventional PCR methods. However, the simple translation of these methods to miRNAs/piRNAs is complicated by the short size of the target, as the length of the primers normally used in the PCR step are as long as mature miRNAs/piRNAs themselves. Shorter primers are typically not useful, as their low duplex melting temperature with the miRNA/piRNAs can introduce signal bias. To avoid these challenges, researchers have developed several of creative approaches based upon the enzymatic modification of conventional primers or altogether new primers for mature miRNAs/piRNAs. Currently, there are two main methods for quantitative PCR of miRNAs/piRNAs (stem-loop RT-based method, and polydenylation based methods).

Statistical Methods:

The receiver operating characteristic (ROC) curve is the standard analytical tool for evaluating diagnostic tests. The diagnostic accuracy of a biomarker is most commonly measured by calculating its sensitivity and specificity. Sensitivity is the proportion of patients who are correctly categorized as having disease among those who truly have the disease. Similarly, specificity is the proportion of patients who are correctly categorized as not having the disease among all patients who truly don't have the disease. Since most diagnostic biomarkers provide results in the continuous scale, the sensitivity and specificity of the test depends on the specific threshold selected. ROC analysis is a statistical method to identify these specific thresholds. The area under the ROC curve (AUC) is the average sensitivity of the biomarker over the range of specificities. It is often used as a summary statistic representing the overall performance of the biomarker. A biomarker with no predictive value would have an AUC of 0.5 (also represented by the diagonal “chance” line above), while a biomarker with perfect ability to predict disease would have an AUC of 1.

### BRIEF DESCRIPTION OF DRAWINGS

FIG. 1 Validation of blood serum piR-5937 (A,B) and piR-28876 (C,D) as diagnostic biomarkers in colorectal cancer. A,C-Mann-Whitney test, B,D-ROC analysis.

FIG. 2 Blood serum levels of piR-5937 are strongly correlated with overall survival of colorectal cancer patients (longrank test, P<0.0001).

FIG. 3 MiRNAs and piRNA levels in colorectal cancer patients (all clinical stages, TNM I-IV) and healthy controls (***, P<0.0001).

FIG. 4 MiRNAs and piRNA levels in colorectal cancer patients (early stages—TNM I a II) and healthy controls.

FIG. 5 Validation of DxScore1 based on miR-23a-3p, miR-27a-3p, miR-142-5p and piR-5937 serum levels in all stages colorectal cancer patients (A,C) and early stages colorectal cancer patients (B,D).

FIG. 6 Validation of DxScore2 based on miR-23a-3p and piR-5937 serum levels in all stages colorectal cancer patients (A,C) and early stages colorectal cancer patients (B,D).

### EXAMPLES

Patients and Methods

Patients

Blood serum samples were collected from 144 colorectal cancer cases (36 patients in TNM stage I, 36 patients in TNM stage II, 36 patients in TNM stage III, 36 patients in TNM stage IV; 82 men, 62 women; mean age 65 years) and 96 controls (48 men, 48 women; mean age 62 years) included in the piRNA profiling study. For the independent validation of candidate piRNAs, another 80 cases (19 patients in TNM stage I, 21 patients in TNM stage II, 21 patients in TNM stage III, 19 patients in TNM stage IV; 44 men, 36 women; mean age 65 years) and 80 controls (47 men, 33 women; mean age 60 years) were included. In addition, another 90 colorectal cancer cases with early stage of the disease (40 patients in TNM stage 1 and 50 patients in TNM stage 2, 50 men, 40 women; mean age 61 years) and 100 controls (52 men, 48 women; mean age 59 years) were used for evaluation of identified miRNA/piRNA biomarkers and models. All subjects enrolled in the study were of the same ethnicity (European descent), and colorectal cancer patients did not receive any neo-adjuvant treatment. Hemolytic serum samples were excluded from the study, as hemolysis may influence the expression of some miRNAs. Written informed consent was obtained from all participants (patients, healthy donors), and the study was approved by the local Ethics Board at Masaryk Memorial Cancer Institute.

RNA Extraction

Before RNA extraction, all samples were checked for hemolysis using the Harboe's spectrophotometric method, which utilizes a 3 point (380 nm, 415 nm, and 450 nm) Allen correction method to quantify hemoglobin concentration. Only the samples with hemoglobin concentration lower than 5 mg·dl−1 were further used in this study. Total RNA enriched for small RNAs was isolated from blood serum using Qiagen miRNeasy Serum/Plasma Kit (Qiagen, GmbH, Hilden, Germany) according to the modified manufacturers' protocol. Briefly, 250 μl of serum was thawed on ice and centrifuged at 14000× g at 4° C. for 5 minutes to remove cellular debris. Subsequently, 200 μl of supernatant was lysed with 1 ml of QIAzol Lysis Reagent. For each sample, 1.25 μl of 0.8 μg·μl−1 MS2 RNA carrier (cat. no. 10165948001, Roche, Basel, Switzerland) was added to 1 ml of QIAzol solution. The extracted RNA was eluted by 2×20 μl of preheated Elution Solution. For the purposes of library preparation, RNA was always isolated separately from 12 samples (12×250 μl; in case of colon cancer patients, all 12 patients were of the same stage. After phase separation, upper aqueous phase from all 12 samples was combined, mixed with 1.5 volume of 100% ethanol and pipetted into one RNeasy MinElute spin column. Elution was performed using 14 μl of preheated Elution Solution. The concentration and purity of RNA were determined spectrophotometrically by measuring its optical density (A260/280>2.0; A260/230>1.8) using NanoDrop ND-1000 Spectrophotometer (Thermo Fisher Scientific, Wilmington, Del., USA). Further, the concentrations and quality of RNA of pooled samples for NGS were also measured using Qubit 2.0 Fluorometer (Thermo Fisher Scientific) and Agilent 2100 Bioanalyzer (Agilent Technologies, Santa Clara, Calif., USA). The samples were either stored at −80° C. or further processed.

Small RNA Library Construction and Sequencing

All libraries were prepared using the Illumina TruSeq Small RNA protocol (Illumina, San Diego, Calif., USA) following the manufacturer's instructions. Briefly, at least 1 μg of total RNA enriched for small RNA was used, and a pair of adaptors was ligated to the 3′ and 5′ ends of piRNAs. Subsequently, 13 cycles of PCR amplification with unique barcode-labeled amplification primers were performed, and size-selection was conducted on 6% native polyacrylamide gel. Then, cDNA fragments between 145 and 160 bp corresponding to the piRNA populations were excised from the gel, eluted and precipitated using ethanol precipitation. The final cDNA pellet was air dried and resuspended in 8 μl of nuclease-free water. The concentration of prepared libraries was measured using High Sensitivity DNA chip and Agilent 2100 Bioanalyzer (Agilent Technologies, Santa Clara, Calif., USA). Equimolar amounts of each library were pooled at a final concentration of 2 nM cDNA, and samples were sequenced on a flowcell with 50-bp single-end reads using MiSeq sequencer (Illumina, San Diego, Calif., USA).

Sequencing Data Processing and Differential piRNA Analyses

Count-based miRNA expression data were generated by the Chimira tool from fastq files. All sequences were adapter trimmed and mapped against piRBase allowing up to two mismatches per sequence. Further analyses were performed using R/Bioconductor packages. piRNAs having less than 1 read per million in more than 17 pooled samples were dropped out. The read counts were pre-normalized by adding normalization factors within edgeR package and further between-sample normalized by the voom function in LIMMA package. After the normalized expression levels were determined, the differentially expressed piRNAs between colorectal cancer patients and healthy controls were screened applying linear model fitting and a Bayes approach. The obtained P values were adjusted for multiple testing using the Benjamini-Hochberg method.

Reverse Transcription and Quantitative Real-Time PCR

Complementary DNA was synthesized from total RNA using gene-specific primers according to the TaqMan MicroRNA Assay protocol (Applied Biosystems, Foster City, Calif., USA). For reverse transcriptase reactions, 10 ng of RNA sample, 50 nM of stem loop RT primer (from miR-23a-3p, miR-27a-3p, miR-142-5p, piR-5937 Applied Biosystems, Thermo Fisher, USA), 1× RT buffer, 0.25 mM each of dNTPs, 3.33 U·μl−1 MultiScribe reverse transcriptase and 0.25 U·μl−1 RNase inhibitor (all from TaqMan MicroRNA Reverse Transcription kit, Applied Biosystems, Foster City, Calif., USA) were used. Reaction mixtures (10 μl) were incubated for 30 min at 16° C., 30 min at 42° C., 5 min at 85° C. and then held at 4° C. (T100TM Thermal Cycler; Bio-Rad, Hercules, Calif., USA). Real-time PCR was performed using the QuantStudio 12K Flex Real-Time PCR system (Applied Biosystems, Foster City, Calif., USA). The 20-μl PCR reaction mixture included 1.33 μl of RT product, 1× TaqMan (NoUmpErase UNG) Universal PCR Master Mix and 1 μl of primer (from miR-23a-3p, miR-27a-3p, miR-142-5p, piR-5937 assays) and probe mix of the TaqMan MicroRNA Assay kit (Applied Biosystems, Foster City, Calif., USA). Reactions were incubated in a 96 well optical plate at 95° C. for 10 min, followed by 40 cycles at 95° C. for 15 s and 60° C. for 1 min.

Data Normalization and Statistical Analyses

The threshold cycle data were calculated by QuantStudio 12K Flex software (Applied Biosystems, Foster City, Calif., USA). All real-time PCR reactions were run in triplicates. The average expression levels of all measured miRNAs were normalized using miR-93-5p (Assay No. 001090; Applied Biosystems, Foster City, Calif., USA) and subsequently analyzed by the 2^{-ΔCt} method. MiR 93-5p was selected as an endogenous control through combination of standard geNorm and NormFinder algorithms (see Vychytilova-Faltejskova et al, 2016). Statistical differences between the levels of analyzed miRNAs in serum samples of colon cancer patients and serum samples of healthy donors were evaluated by two-tailed non-parametric Mann-Whitney test. Paired samples before and after surgery were analyzed using two-tailed non-parametric Wilcoxon test for paired samples. Analysis of Kaplan-Meier survival curves and longrank test was performed to evaluate prognostic potential of piR-5937. All calculations were performed using GraphPad Prism version 5.00 (GraphPad Software, La Jolla, Calif., USA) and R environment (R Development Core Team). P-values of less than 0.05 were considered statistically significant.

### Example 1

PIWI-Interacting RNAs with Differential Levels in Body Fluid of Colorectal Cancer Patients and Healthy Donors

Small RNA sequencing of RNA purified from blood serum samples from 144 colorectal cancer patients and 96 healthy controls was carried out using MiSeq sequencer (Illumina). In total, 20 small RNA libraries were prepared and sequenced (12 libraries per colon cancer patients, 8 libraries per healthy controls, RNA samples from 12 patients/healthy donors in each library). On average, more than 96% of the reads had Q-score higher than 30, thus the obtained data were considered to be of high quality. The sequenced samples contained on average 8,925,000±2,139,597 reads and 8,219,664±1,954,177 reads passed the filter. In total, 470 and 453 piRNAs were detectable (more than 50 copies per 1 million of reads) in colorectal cancer patients and healthy donors, respectively. From these, 39 miRNAs were found to have significantly different levels (10 up-regulated, 29 down-regulated; Table 1) in serum samples of colorectal cancer patients compared to healthy donors (adjusted P<0.005).

TABLE 1
Summary of 39 piRNAs with significantly different levels
in blood serum of colorectal cancer patients and healthy
donors (adjusted P < 0.005)

| | | Change | | |
|---|---|---|---|---|
| | | with | | |
| PiRNA | Sequence | CRC | log FC | p-value |
| piR-hsa-32161 | CCCCCCACTGCTAAATTTGACTGG (SEQ ID NO. | DOWN | −1.208 | 6.232 · 10⁻⁴ |
| | 6) | | | |
| piR-hsa-32238 | CCCCCACTGCTAAATTTGACTGGTT (SEQ ID NO. | DOWN | −1.114 | 3.002 · 10⁻³ |
| | 7) | | | |
| piR-hsa-1242 | AGCCCGGCTAGCTCAGTCGGTAGAGCATGAG | DOWN | −1.203 | 4.31 · 10⁻⁴ |
| | (SEQ ID NO. 8) | | | |
| piR-hsa-32187 | CCCCCCACTGCTAAATTTGACTG (SEQ ID NO. 9) | DOWN | −1.173 | 2.292 · 10⁻³ |
| piR-hsa-27620 | GCCCGGCTAGCTCAGTCGGTAGAGCATGAG (SEQ | DOWN | −1.081 | 1.678 · 10⁻³ |
| | ID NO. 10) | | | |
| piR-hsa-32162 | CCCCCCACTGCTAAATTTGACTGGC (SEQ ID NO. | DOWN | −1.567 | 3.98 · 10⁻⁶ |
| | 11) | | | |
| piR-hsa-32195 | CCCCCCACTGCTAAATTTGACTGGT (SEQ ID NO. | DOWN | −1.048 | 1.0366 · 10⁻³ |
| | 12) | | | |
| piR-hsa-32158 | CCCCCCACTGCTAAATTTGACTGGT (SEQ ID NO. | DOWN | −2.021 | 2.1181 · 10⁻³ |
| | 13) | | | |
| piR-hsa-29218 | TAAAGTGCTGACAGTGCAGATAGTGGTCCTC | DOWN | −1.093 | 1.1209 · 10⁻³ |
| | (SEQ ID NO. 14) | | | |
| piR-hsa-28876 | GTTTCCGTAGTGTAGTGGTCATCACGTTCGC (SEQ | DOWN | −2.367 | 8.51 · 10⁻⁹ |
| | ID NO. 15) | | | |
| piR-hsa-5937 | TCCCTGGTGGTCTAGTGGTTAGGATTCGGCA (SEQ | DOWN | −2.999 | 8.46 · 10⁻¹² |
| | ID NO. 1) | | | |
| piR-hsa-24672 | TTCCCTGGTGGTCTAGTGGTTAGGATTCGGC (SEQ | DOWN | −3.130 | 4.03 · 10⁻¹¹ |
| | ID NO. 16) | | | |
| piR-hsa-26872 | GAGGAATGATGACAAGAAAAGGCCGAA (SEQ ID | UP | 1.149 | 2.6204 · 10⁻³ |
| | NO. 17) | | | |
| piR-hsa-8226 | TCTGCTGCCTCAGCCTCCCGAGTAGCTGA (SEQ ID | UP | 1.910 | 7.435 · 10⁻⁴ |
| | NO. 18) | | | |
| piR-hsa-30715 | TACTCAGGAGGCTGAGGCAGGAGAATGGC (SEQ | UP | 1.320 | 3.304 · 10⁻³ |
| | ID NO. 19) | | | |
| piR-hsa-6746 | TCCTGGGTTCAGGTGATTCTCCTGCCTCAGT (SEQ | UP | 1.693 | 2.4257 · 10⁻³ |
| | ID NO. 20) | | | |
| piR-hsa-30937 | TAGTCCCAGCTACTTGGGAGGCTGAGGCA (SEQ | UP | 1.917 | 0.0001423 |
| | ID NO. 21) | | | |
| piR-hsa-26543 | GACCAGCCTGGCCAACATGGTGAAACCCCGCC (SEQ | UP | 1.597 | 2.5176 · 10⁻³ |
| | ID NO. 22) | | | |
| piR-hsa-15278 | TGCCTCCCGGATTCAAGTGATTCTCCTGCCT (SEQ | UP | 1.849 | 9.70 · 10⁻⁵ |
| | ID NO. 23) | | | |
| piR-hsa-5505 | TCCCAGCTACCTAGGAGGCTGAGGCAGGAG (SEQ | UP | 1.903 | 1.464 · 10⁻⁴ |
| | ID NO. 24) | | | |
| piR-hsa-30714 | TACTCAGGAGGCTGAGACAGGAGAATTGC (SEQ | UP | 1.574 | 9.617 · 10⁻⁴ |
| | ID NO. 25) | | | |
| piR-hsa-1359 | ATCGAGGCTAGAGTCACGCTTGGGTATCGGCT | DOWN | −1.359 | 2.6683 · 10⁻³ |
| | (SEQ ID NO. 26) | | | |
| piR-hsa-27139 | GCCTGGGTAGCTCAGTCGGTAGAGCATCAGA | DOWN | −1.341 | 4.7537 · 10⁻³ |
| | (SEQ ID NO. 27) | | | |
| piR-hsa-28488 | GGTCGCTGGTTCGAATCCGGCTCGAAGGACC | DOWN | −1.443 | 2.1103 · 10⁻³ |
| | (SEQ ID NO. 28) | | | |
| piR-hsa-27616 | GCCCGGATGATCCTCAGTGGTCTGGGGTGC (SEQ | DOWN | −1.277 | 4.8853 · 10⁻³ |
| | ID NO. 29) | | | |
| piR-hsa-29716 | TAAGGTGCATCTAGTGCAGATAGTGAAGTA (SEQ | DOWN | −1.323 | 3.3901 · 10⁻³ |
| | ID NO. 30) | | | |
| piR-hsa-28019 | GGAGGTGATGAACTGTCTGAGCCTGACC (SEQ ID | DOWN | −1.945 | 3.2583 · 10⁻³ |
| | NO. 31) | | | |
| piR-hsa-23231 | CCCCTGGTGGTCTAGTGGTTAGGATTCGGC (SEQ | DOWN | −1.855 | 9.07 · 10⁻⁵ |
| | ID NO. 32) | | | |
| piR-hsa-28846 | GTTCACTGATGAGAGCATTGTTCTGAGCCA (SEQ | DOWN | −2.050 | 2.142 · 10⁻⁴ |
| | ID NO. 33) | | | |
| piR-hsa-24360 | TTCACTGATGAGAGCATTGTTCTGAGC (SEQ ID | DOWN | −1.430 | 4.8228 · 10⁻³ |
| | NO. 34) | | | |
| piR-hsa-6046 | TCCGATCCTCGTTGTTTTGGCTATGGCCAGA (SEQ | DOWN | −1.396 | 1.4122 · 10⁻³ |
| | ID NO. 35) | | | |
| piR-hsa-23940 | CTGACCTCAAGTGATCCACCTGCCTCAGCC (SEQ | UP | 1.217 | 3.495 · 10⁻³ |
| | ID NO. 36) | | | |
| piR-hsa-32182 | CCCCCACTGCTAAATTTGACTGGCTA (SEQ ID NO. | DOWN | −1.044 | 2.0513 · 10⁻³ |
| | 37) | | | |
| piR-hsa-32167 | CCCCCACTGCTAAATTTGACTGGCT (SEQ ID NO. | DOWN | −1.108 | 1.2705 · 10⁻³ |
| | 38) | | | |
| piR-hsa-28190 | GGCCGTGATCGTATAGTGGTTAGTACTCTG (SEQ | DOWN | −1.277 | 7.75 · 10⁻⁵ |
| | ID NO. 39) | | | |
| piR-hsa-23210 | CCCCCCACTGCTAAATTTGACTGGTT (SEQ ID NO. | DOWN | −1.418 | 3.86 · 10⁻⁵ |
| | 40) | | | |
| piR-hsa-11362 | TGAGGTAGTAGGTTGTATGGTTTAGAGTTACA | DOWN | −0.890 | 3.728 · 10⁻³ |
| | (SEQ ID NO. 41) | | | |
| piR-hsa-23209 | CCCCCCACTGCTAAATTTGACTGGCTA (SEQ ID | DOWN | −1.642 | 3.79 · 10⁻⁵ |
| | NO. 42) | | | |
| piR-hsa-32159 | CCCCCCACTGCTAAATTTGACTGGCT (SEQ ID NO. | DOWN | −1.723 | 3.02 · 10⁻⁵ |
| | 43) | | | |

Validation of Candidate piRNAs in Independent Cohorts of Patients and Controls

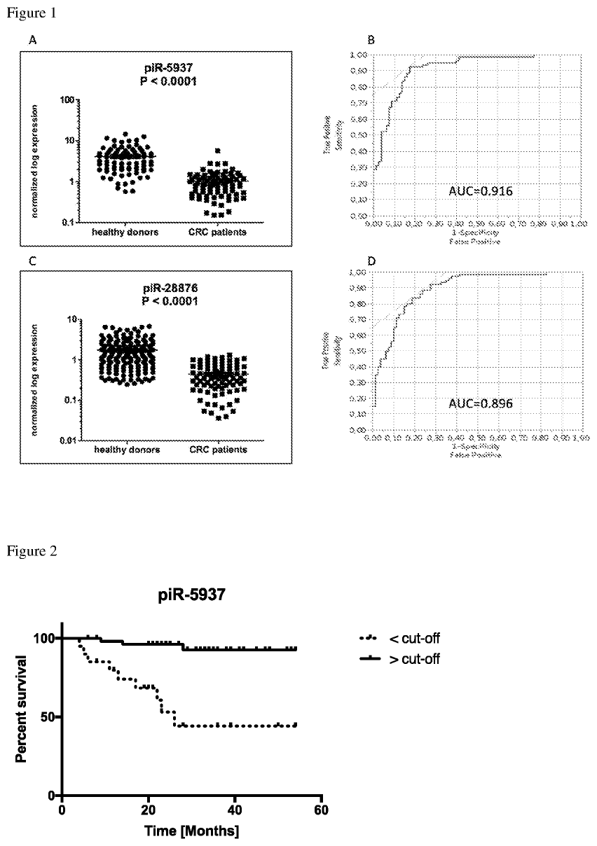

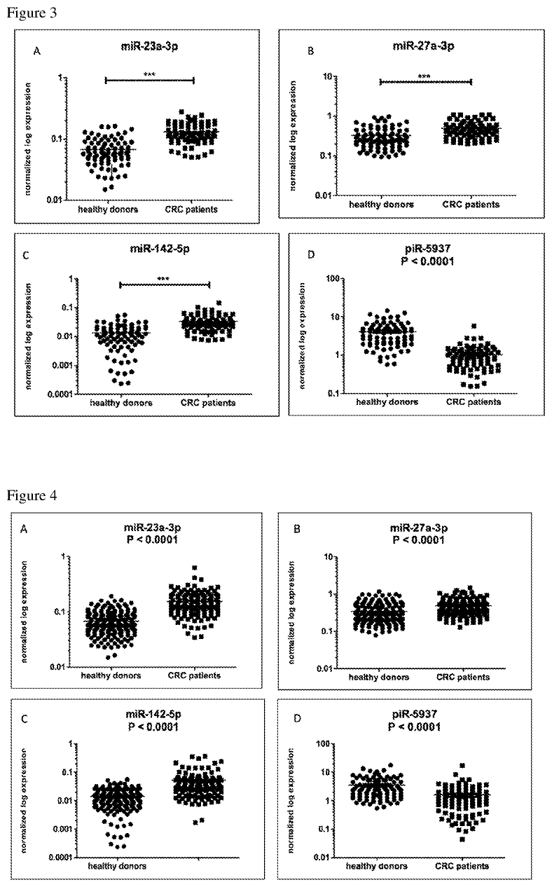

In total, serum samples from 80 colorectal cancer patients and 80 healthy donors were included in the validation phase of the study. Out of 39 piRNAs identified by smallRNA sequencing to have significantly lower levels in blood serum of colorectal cancer patients in comparison to healthy controls, piR-5937 and piR-28876 were selected as the most promising for independent validation. Both piRNAs were proved to be significantly decreased in blood serum of colorectal cancer patients compared to healthy donors: piR-5937 (P<0.0001, AUC=0.916, sensitivity=92.5%, specificity=82.5%), piR-28876 (P<0.0001, AUC=0.896, sensitivity=92.5%, specificity=72.5%) (see FIG. 1). Moreover, higher levels of blood serum piR-5937 were significantly associated with more favourable survival than those with lower levels (P<0.0001; HR 0.09, CI 0.02-0.036, FIG. 2), which shows its prognostic effect.

### Example 2 Development and Validation of Colorectal Cancer Diagnostic Models (DxScores) Based on Combination of Blood Serum piRNAs and miRNAs Levels

The development of colorectal cancer diagnostic models (DxScores) based on piRNA/miRNA levels in blood serum was performed using logistic regression (for diagnosis). To generate diagnosis-related signature, piR-5937, piR-28876 and the top 4 miRNAs identified in the study of Vychytilova-Faltejskova et al. (2016, cited in the Background Art chapter) (miR-23a-3p, miR-27a-3-, miR-142-5p, miR-376c-3p) were introduced into a bidirectional stepwise logistic regression model on the training set of samples. The final model was taken as that which maximizes the Akaike information criterion. A ROC (receiver operating characteristic) curve was generated for further analysis of the DXscores. The maximum Youden index was used to obtain optimal cut-off value for discrimination of healthy donors and colon cancer patients. Subsequently, ROC curve analysis for each set of samples was applied to calculate the sensitivity and specificity of DXscore. Validation of the obtained model and cut-off was performed on the independent validation set of samples with early stages of the disease. This approach led to establishment of the two efficient diagnostic models: (i) based on the expression of 4 biomarkers (miR-23a-3p, miR-27a-3p, miR-142-5p and piR-5937) (DxScore1) and (ii) based on the expression of 2 biomarkers (miR-23a-3p and piR-5937) (DxScore2). Both were characterized by very high sensitivity and specificity in diagnosis of colorectal cancer from blood serum specimen. All miRNAs/piRNA included in the DxScores indicated significantly different levels in blood serum of colorectal cancer patients and healthy donors. Levels of miRNAs were higher and levels of piRNA were lower in blood serum of patients in both validation cohorts (1^{st} cohort—all TNM stages, 2^{nd} cohort—early TNM stages) (FIGS. 3 and 4).

The best analytical performance indicated a model based on 4 biomarkers (miR-23a-3p, miR-27a-3p, miR-142-5p and piR-5937) (DxScore 1). Diagnostic score was calculated according to the following formula:

DxScore1=−0,8895+62,92648*miR-23a−8,08928*miR-27a+73,16745*miR-142-5p−1,6757*piR-5937

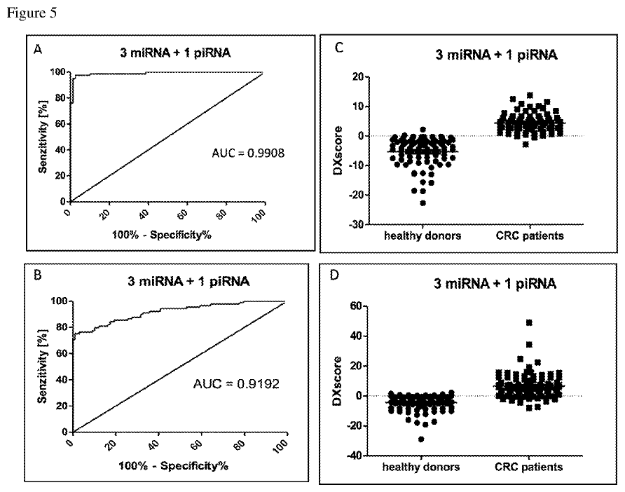

DxScore1 enabled to identify colorectal cancer with sensitivity 99% and specificity 90% in cohort of patients with all TNM stages (FIG. 5A,C). If the same DxScore1 and cut-off value was applied in independent validation cohort of patients with early TNM stages of the disease, sensitivity was 84% and specificity 83% (FIG. 5B,D), which significantly outperforms current diagnostic approaches used in colorectal cancer (summarized in Table 2).

TABLE 2
Summary of analytical performance of DxScore1
in diagnosis of colorectal cancer.

| | Validation phase | Validation phase I + II |
|---|---|---|
| | (TNM stage I − IV) | (TNM stage I + II) |
| AUC | 0.9908 | 0.9192 |
| sensitivity | 96.25% | 84.27% |
| specificity | 97.50% | 82.65% |
| accuracy | 96.86% | 83.46% |
| PPV | 90.70% | 81.72% |
| NPV | 97.30% | 85.26% |
| cut-off | −0.4799 | −0.4799 |

The slightly lower analytical performance in comparison to DxScore1 indicated a model based on 2 biomarkers (miR-23a-3p and piR-5937) (DxScore2). Diagnostic score was calculated according to the following formula:

DxScore2=−0,64243+39,96723*miR-23a-3p−1,62861*piR-5937

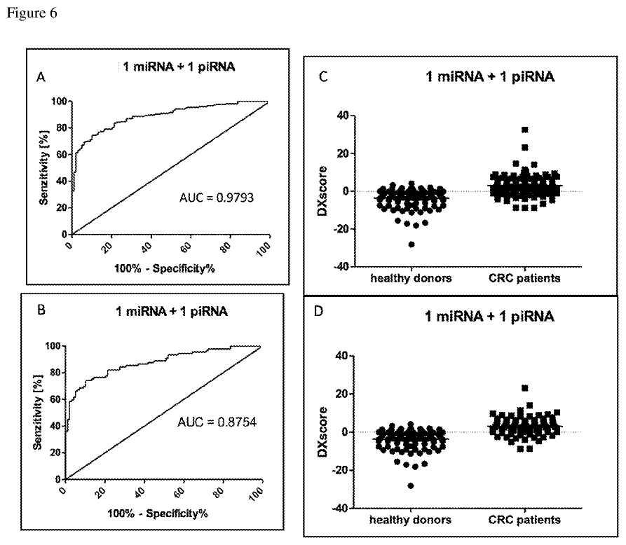

DxScore2 enabled to identify colorectal cancer with sensitivity 94% and specificity 90% in cohort of patients with all TNM stages (FIG. 6A,C). If the same DxScore2 and cut-off value was applied in independent validation cohort of patients with early TNM stages of the disease, sensitivity was 82% and specificity 75% (FIG. 6B,D), which is still satisfactory performance when compared to current diagnostic approaches used in colorectal cancer (summarized in Table 3).

TABLE 3
Summary of analytical performance of DxScore2
in diagnosis of colorectal cancer.

| | Validation phase | Validation phase |
|---|---|---|
| | (TNM stage I − IV) | (TNM stage I + II) |
| AUC | 0.9793 | 0.8754 |
| sensitivity | 93.67% | 82.01% |
| specificity | 90.00% | 75.51% |
| accuracy | 91.82% | 78.69% |
| PPV | 90.24% | 74.00% |
| NPV | 92.31% | 81.82% |
| cut-off | −0.09549 | −0.09549 |

1. A method for diagnosing colorectal cancer, comprising the steps of administering an anti-cancer treatment to a patient in whose body fluid sample the colorectal cancer is diagnosed, characterized in that a level of expression of piRNA-hsa-5937, having the sequence TCCCTGGTGGTCTAGTGGTTAGGATTCGGCA (SEQ ID NO. 1),
and levels of expression in the body fluid sample of at least one miRNA selected from:
(SEQ ID NO. 3)
miR-23a-3p, having the sequence
AUCACAUUGCCAGGGAUUUCC,
(SEQ ID NO. 4)
miR-27a-3p, having the sequence
UUCACAGUGGCUAAGUUCCGC,
(SEQ ID NO. 5)
miR-142-5p, having the sequence
CAUAAAGUAGAAAGCACUACU
are determined in the body fluid sample,
then the level of expression of piRNA-hsa-5937 and of the at least one miRNA in the sample is compared with levels of expression of piRNA-hsa-5937 and of the at least one miRNA in the body fluid of a healthy human,
and
when the level of expression of piRNA-hsa-5937 in the sample is lower than the level of expression of piRNA-hsa-5937 in the body fluid of a healthy human, and
when the level of expression of said at least one miRNA in the sample is higher than the level of expression of said miRNAs in body fluid of a healthy human,
colorectal cancer is diagnosed in the sample.

2. The method according to claim 1, wherein the levels of expression of all of
piRNA-hsa-5937,
miR-23a-3p,
miR-27a-3p,
miR-142-5p,
are determined in a sample of body fluid.

3. The method according to claim 1, which further includes determining of levels of expression in the body fluid sample of at least one of the following piRNAs, comparing them with the levels of expression of the same piRNA, respectively, in body fluid of a healthy human, whereas when the level of expression of said piRNA shows the change determining colorectal cancer relative to the level of expression of the same piRNA in body fluid of a healthy human, colorectal cancer is diagnosed in the sample:

| | | Change in |
|---|---|---|
| | | expression |
| | | level |
| | | determining |
| | | colorectal |
| piRNA | Sequence | cancer |
| piR-hsa-32161 | CCCCCCACTGCTAAATTTGACTGG (SEQ ID NO. 6) | decrease |
| piR-hsa-32238 | CCCCCACTGCTAAATTTGACTGGTT (SEQ ID NO. 7) | decrease |
| piR-hsa-1242 | AGCCCGGCTAGCTCAGTCGGTAGAGCATGAG (SEQ ID NO. 8) | decrease |
| piR-hsa-32187 | CCCCCCACTGCTAAATTTGACTG (SEQ ID NO. 9) | decrease |
| piR-hsa-27620 | GCCCGGCTAGCTCAGTCGGTAGAGCATGAG (SEQ ID NO. 10) | decrease |
| piR-hsa-32162 | CCCCCCACTGCTAAATTTGACTGGC (SEQ ID NO. 11) | decrease |
| piR-hsa-32195 | CCCCCCACTGCTAAATTTGACTGGT (SEQ ID NO. 12) | decrease |
| piR-hsa-32158 | CCCCCCACTGCTAAATTTGACTGGT (SEQ ID NO. 13) | decrease |
| piR-hsa-29218 | TAAAGTGCTGACAGTGCAGATAGTGGTCCTC (SEQ ID NO. 14) | decrease |
| piR-hsa-28876 | GTTTCCGTAGTGTAGTGGTCATCACGTTCGC (SEQ ID NO. 15) | decrease |
| piR-hsa-24672 | TTCCCTGGTGGTCTAGTGGTTAGGATTCGGC (SEQ ID NO. 16) | decrease |
| piR-hsa-26872 | GAGGAATGATGACAAGAAAAGGCCGAA (SEQ ID NO. 17) | increase |
| piR-hsa-8226 | TCTGCTGCCTCAGCCTCCCGAGTAGCTGA (SEQ ID NO. 18) | increase |
| piR-hsa-30715 | TACTCAGGAGGCTGAGGCAGGAGAATGGC (SEQ ID NO. 19) | increase |
| piR-hsa-6746 | TCCTGGGTTCAGGTGATTCTCCTGCCTCAGT (SEQ ID NO. 20) | increase |
| piR-hsa-30937 | TAGTCCCAGCTACTTGGGAGGCTGAGGCA (SEQ ID NO. 21) | increase |
| piR-hsa-26543 | GACCAGCCTGGCCAACATGGTGAAACCCCGCC (SEQ ID NO.22) | increase |
| piR-hsa-15278 | TGCCTCCCGGATTCAAGTGATTCTCCTGCCT (SEQ ID NO. 23) | increase |
| piR-hsa-5505 | TCCCAGCTACCTAGGAGGCTGAGGCAGGAG (SEQ ID NO. 24) | increase |
| piR-hsa-30714 | TACTCAGGAGGCTGAGACAGGAGAATTGC (SEQ ID NO. 25) | increase |
| piR-hsa-1359 | ATCGAGGCTAGAGTCACGCTTGGGTATCGGCT (SEQ ID NO.26) | decrease |
| piR-hsa-27139 | GCCTGGGTAGCTCAGTCGGTAGAGCATCAGA (SEQ ID NO. 27) | decrease |
| piR-hsa-28488 | GGTCGCTGGTTCGAATCCGGCTCGAAGGACC (SEQ ID NO. 28) | decrease |
| piR-hsa-27616 | GCCCGGATGATCCTCAGTGGTCTGGGGTGC (SEQ ID NO. 29) | decrease |
| piR-hsa-29716 | TAAGGTGCATCTAGTGCAGATAGTGAAGTA (SEQ ID NO. 30) | decrease |
| piR-hsa-28019 | GGAGGTGATGAACTGTCTGAGCCTGACC (SEQ ID NO. 31) | decrease |
| piR-hsa-23231 | CCCCTGGTGGTCTAGTGGTTAGGATTCGGC (SEQ ID NO. 32) | decrease |
| piR-hsa-28846 | GTTCACTGATGAGAGCATTGTTCTGAGCCA (SEQ ID NO. 33) | decrease |
| piR-hsa-24360 | TTCACTGATGAGAGCATTGTTCTGAGC (SEQ ID NO. 34) | decrease |
| piR-hsa-6046 | TCCGATCCTCGTTGTTTTGGCTATGGCCAGA (SEQ ID NO. 35) | decrease |
| piR-hsa-23940 | CTGACCTCAAGTGATCCACCTGCCTCAGCC (SEQ ID NO. 36) | increase |
| piR-hsa-32182 | CCCCCACTGCTAAATTTGACTGGCTA (SEQ ID NO. 37) | decrease |
| piR-hsa-32167 | CCCCCACTGCTAAATTTGACTGGCT (SEQ ID NO. 38) | decrease |
| piR-hsa-28190 | GGCCGTGATCGTATAGTGGTTAGTACTCTG (SEQ ID NO. 39) | decrease |
| piR-hsa-23210 | CCCCCCACTGCTAAATTTGACTGGTT (SEQ ID NO. 40) | decrease |
| piR-hsa-11362 | TGAGGTAGTAGGTTGTATGGTTTAGAGTTACA (SEQ ID NO.41) | decrease |
| piR-hsa-23209 | CCCCCCACTGCTAAATTTGACTGGCTA (SEQ ID NO. 42) | decrease |
| piR-hsa-32159 | CCCCCCACTGCTAAATTTGACTGGCT (SEQ ID NO. 43) | decrease |

4. The method according to claim 1, wherein the level of expression of at least one diagnostic marker piRNA or miRNA in the sample is normalized to a substance having a constant content in the body fluid, preferably to miR-93-5p, having the sequence CAAAGUGCUGUUCGUGCAGGUAG (SEQ ID NO. 2).

5. The method according to claim 1, wherein the level of expression of at least one diagnostic marker piRNA or miRNA in the sample is compared to a pre-determined cut-off value, preferably obtained using a statistical analysis of the levels of expression, optionally normalized levels of expression, in groups of healthy controls and colorectal cancer patients.

6. The method according to claim 2, wherein the levels of expression of at least two diagnostic marker piRNA or miRNA in the sample are used to calculate a diagnostic score, which is then compared to a pre-determined cut-off value, preferably obtained using a statistical analysis of the levels of expression, optionally normalized levels of expression, in groups of healthy controls and colorectal cancer patients.

7. A method of prognosis of overall survival of the patient, characterized in that a level of expression of piRNA-hsa-5937 is determined in a sample of body fluid, then the level of expression of piRNA-hsa-5937 is compared with a reference expression level, whereas the level of expression of piRNA-hsa-5937 in the sample which is higher than the reference expression level indicates a favourable overall survival prognosis, while the level of expression of piRNA-hsa-5937 in the sample which is lower than the reference expression level indicates a poor overall survival prognosis, wherein the reference expression level is obtained from a statistical analysis of samples from a group of colorectal cancer patients with favourable overall survival time and a group of colorectal cancer patients with poor overall survival time.

8. The method according to claim 7, wherein the level of expression of piRNA-hsa-5937 in the sample is referenced to a substance having a constant level of expression in the body fluid, such as miR-93-5p.

9. The method according to claim 1, wherein the body fluid is selected from blood, blood serum, blood plasma, urine and saliva.
